(19) United States
(12) Patent Application Publication
(10) Pub. No.: US 2017/0209503 A1
(43) Pub. Date: Jul. 27, 2017

(54) PRODUCTION OF BEADLETS COMPRISING PROBIOTIC COMPOUNDS

(71) Applicant: DSM IP ASSETS B.V., Heerlen (NL)

(72) Inventors: Elger FUNDA, Grenzach-Wyhlen (DE); Karin LEUTHARDT, Basel (CH)

(21) Appl. No.: 15/481,332

(22) Filed: Apr. 6, 2017

Related U.S. Application Data
(63) Continuation of application No. 13/057,212, filed on Sep. 16, 2011, now abandoned.

(30) Foreign Application Priority Data
Aug. 4, 2008 (EP) 08161730.0

(51) Int. Cl. A61K 35/741; C12N 1/20; A23K 10/18; A23K 20/163; A23P 10/30; A23K 50/10; A23K 50/30; A23K 50/75; A23L 33/135; A23L 33/125; A61K 9/16; A23K 40/30

(52) U.S. Cl. CPC A61K 35/741; A61K 9/1682; A61K 9/1652; C12N 1/20; A23K 10/18; A23K 20/163; A23K 40/30; A23K 50/10; A23K 50/30; A23K 50/75; A23L 33/135; A23L 33/125; A23P 10/30; A23V 2002/00

(57) ABSTRACT

The present invention relates a process of production of beadlets comprising probiotic compounds in a matrix comprising at least one starch and/or starch derivative, to such beadlets and to the use of such specific beadlets in food (for humans and animals) as well as in premixes.

This application is a continuation of commonly owned U.S. application Ser. No. 13/057,212, filed Sep. 16, 2011 (now abandoned), which is the U.S. national phase of International Application No. PCT/EP2009/059957, filed Jul. 31, 2009, which designated the U.S. and claims priority to European Application No. 08161730.0, filed Aug. 4, 2008, the entire contents of each of which are hereby incorporated by reference.

The present invention relates to a process of production of beadlets comprising probiotic compounds in a matrix, which comprise at least one starch and/or starch derivative, to such beadlets and to the use of such specific beadlets.

The term “probiotic” generally refers to a non-pathogenic bacterium fed to animals, including birds, as a way to prevent colonization by pathogenic microorganisms, e.g. protazoa. Probiotics may also be defined as live, or livable, micro-organisms which beneficially affect the intestinal balance of healthy and normally functioning humans and animals.

Probiotics are delivered to farm animals for improving their intestinal microbial balance but also to human in the form of dairy-based foods containing intestinal species of lactobacilli and bifidobacteria.

Probiotics as live microorganisms are sensible to treat, store and formulate. Therefore the formulation of probiotics can be challenging.

The most common commercial formulation for probiotics are dairy products and probiotic fortified food (as well feed) products. However, tablets, capsules, powders and sachets containing the bacteria in freeze dried form are also available. Probiotics are also used in animal nutrition.

A very typical form of formulation of substances used in consumer products (such as for example food products) are powders. Powders can be produced by spray drying or freeze-drying processes. Freeze-drying is commonly used for drying of probiotics. Nevertheless this process is time-consuming and expensive. Moreover, viability of the bacteria may be negatively affected by the drying step.

Spray-drying is also used for drying of probiotics. This process is economically favorable but has certain disadvantages in the case of probiotics:
Usually high temperatures (>60° C.) are used in the production process. This has a negative effect on the activity of the probiotics.
Using a low-temperature spray-drying processes with product temperatures <65° C. usually leads to products with a relatively high content of residual water. Therefore, the storage stability of such powders is usually not sufficient.
Powders are usually dusty and therefore their handling can be difficult.

Another well known form of preparations are beadlets. Beadlets provide superior handling properties in that they are not dusty and possess good flowability characteristics. Beadlets are solely known for fat-soluble substances.

Beadlets (comprising fat-soluble substances) and their methods of productions are known from the prior art. These beadlets comprise fat-soluble (lipophilic, hydrophobic) substances. Such beadlets and their process for production are for example known from US2006/0115534 and U.S. Pat. No. 4,670,247. These beadlets usually have good storage stability, but the concentration of the fat-soluble substances in such beadlets is low. Usually the content is between 5-15 weight-% (wt-%), based on the total weight of the beadlet.

Another disadvantage is that the production of such beadlets requires an emulsification or dispersion step to distribute the water-insoluble active in the aqueous matrix phase. Therefore, either the matrix material needs to have emulsifying properties or an additional emulsifier is required.

The goal of the present invention was to find a process for producing formulations of probiotics, which
allows producing formulations with a high amount of probiotic bacteria,
produces storage stable formulations, and
is a simple process.

Surprisingly, it has been found that the use of the powder catch process allows producing such formulations, which are in the form of beadlets having the above mentioned advantages. The beadlets comprise at least one probiotic compound and at least one starch and/or at least one starch derivative as a matrix material. Furthermore, these beadlets are coated with a layer of the powder catch medium.

Therefore, the present invention relates to a process for preparing beadlets, which comprise at least one probiotic compound, comprising:
(a) forming an aqueous solution of
   (i) at least one probiotic compound and
   (ii) at least one starch and/or at least one starch derivative,
(b) converting the solution into a dry powder by spray drying into a collecting powder.

The principle of such a process is known from the prior art. It can be found for example in U.S. Pat. No. 6,444,227 or WO04062382. These references are hereby incorporated by reference.

Probiotic compounds in the context of the present invention are dietary supplements containing potentially beneficial bacteria or yeasts. According to the currently adopted definition by FAO/WHO, probiotics are: ‘Live microorganisms which when administered in adequate amounts confer a health benefit on the host’. Lactic acid bacteria (LAB) are the most common type of microbes used. LAB have been used in the food industry for many years, because they are able to convert sugars (including lactose) and other carbohydrates into lactic acid. This not only provides the characteristic sour taste of fermented dairy foods such as yogurt, but also by lowering the pH may create fewer opportunities for spoilage organisms to grow, hence creating possible health benefits on preventing gastrointestinal infections. Strains of the genera Lactobacillus and Bifidobacterium are the most widely used probiotic bacteria. Propionibacteria and other bacteria are also used as probiotics. Probiotic bacterial cultures are intended to assist the body's naturally occurring gut flora, an ecology of microbes, to re-establish themselves. In animal nutrition, probiotics are intended to have beneficial effects on animal health. In the case of dairy cows, probiotics are intended to increase milk production and milk quality.

Probiotic bacterial cultures are for example Debaromyces, Candida, Pichia and Torulopsis, moulds such as Aspergillus, Rhizopus, Mucor, and Penicillium and Torulopsis and bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus. Specific examples of suitable probiotic micro-organisms (probiotic compounds) are: Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Laciobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus reuteri, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus, and Staphylococcus xylosus.

Preferred probiotic compounds used in functional food are lactic acid bacteria, mainly lactobacilli. Lactobacilli are non-pathogenic micro-organisms, colonizing the human intestinal and urogenital tract from early childhood to old age. Nowadays, several commercial probiotic lactobacilli are successfully used, among which Lactobacillus rhamnosus is one of the best known. Several strains of Lactobacillus fermentum are used for correction and stabilization of intestinal micro-flora in case of dysbacterioses and urogenital infections with different ethiologies.

Preferred examples of probiotics used as feed additive for farm animals are strains from the genera of Bacillus, Lactobacillus, Pediococcus and Propionibacterium.

A preferred strain of Bacillus is a strain of Bacillus licheniformis, preferably selected from the following strains of Bacillus licheniformis: ATCC 14580 (=NCIB 9375), NCIMB 6346 (=DSM 8785), NCTC 1024, NCTC 1025, NCTC 2120, NCTC 7589, NCTC 9932, ATCC 21424, NCIMB 10689, and ATCC 53757. A preferred subgroup includes Bacillus licheniformis ATCC 14580 (=NCIB 9375), and Bacillus licheniformis NCIMB 6346 (=DSM 8785).

A preferred strain of Lactobacillus is a strain of Lactobacillus reuteii, Lactobacillus acidophilus, Lactobacillus animalis, Lactobacillus ruminis, Lactobacillus johnsonii, Lactobacillus casei, Lactobacillus paracasei, Lactobacillus rhamnosus, and Lactobacillus fermentum. In a preferred embodiment the probiotic strain is selected from the group consisting of Lactobacillus reuteri (NCC2581; CNCM I-2448), Lactobacillus reuteri (NCC2592; CNCM I-2450), Lactobacillus rhamnosus (NCC2583; CNCM I-2449), Lactobacillus reuteri (NCC2603; CNCM I-2451), Lactobacillus reuteri (NCC2613; CNCM I-2452), and Lactobacillus acidophilus (NCC2628; CNCM I-2453).

More preferred is a process wherein the microorganism is of the genus Propionibacterium (P.) and more preferably P. acidipropionici and P. jensenii. Preferred strains of bacteria include P. acidipropionici and P. jensenii strains P169, P170, P179, P195, and P261, especially strain P169. The strains P 169 and P170 are available from the microorganism collection of the American Type Culture Collection (ATCC), 10801 University Blvd., Manassas, Va., 20110, under accession numbers ATCC PTA-5271 and ATCC PTA-5272, respectively, which have been deposited on Jun. 18, 2003.

It is at present contemplated that the probiotic strain is administered in one or more of the following amounts (dosage ranges): 10 E2-14, 10 E4-12, 10 E6-10, 10 E7-9, preferably 10 E8 CFU/g of final feed (the designation E meaning exponent, viz., e.g., 10 E2-14 means 10̂2-10̂14).

CFU (Colony Forming Units) is defined as one or more, sometimes many, bacterial cells that grow into a visible colony on a Petri dish containing appropriate agar for the microorganism being tested. One colony is one CFU. For instance, 1 CFU may contain 20 bacterial cells or just one cell.

In a preferred embodiment, the beadlets comprising the microorganism are fed to a ruminant, and the microorganism becomes established in the rumen. Preferably, the amount of the microorganism that is delivered to the ruminant is about 6×10⁹ CFU to about 6×10¹² CFU/animal/day. This translates into approximately 1×10⁵ to 1×10⁸ CFU/ml of rumen fluid for an averaged sized cow. In a more preferred embodiment, about 6×10¹¹ CFU/animal/day of the microorganism is delivered to the ruminant. In a preferred embodiment, the microorganism is fed to a ruminant such that the microorganism becomes established in the rumen at a level of about 1×10⁵ CFU per ml of rumen fluid to about 1×10⁸ CFU per ml of rumen fluid.

The probiotic compounds are formulated in a beadlet by a matrix material, which comprises at least one starch and/or at least one starch derivative.

Starch having the chemical formula (C₆H₁₀O₅)ₙ is a polysaccharide carbohydrate consisting of a large number of glucose monosaccharide units joined together by glycosidic bonds. All plant seeds and tubers contain starch. Starches are commonly extracted from plants, such as corn, sorghum, wheat, rice, tapioca, arrowroot, sago, potato, quinoa and amaranth. Natural starches contain usually amylase and amylopectin molecules. The content of amylase in natural starches can vary from 0 wt-% (for example waxy corn starch and waxy rice starch) up to about 85 wt-% (High amylase corn starch). Normal starches contain about 25 wt-% of amylase. As a consequence thereof the content of amylopectin is between 15 wt-% and 100 wt-%.

It is also possible to use starch derivatives (modified starches) including hydrolyzed starches. The starches can be modified in various manners. It can be done physically and chemically.

Pregelatinised starches are examples of physically modified starches.

Acidic modified, oxidized, cross-linked, starch esters, starch ethers and cationic starches are examples of chemically modified starches. Important examples of such modified starches are octenyl succinic anhydride starches (OSA starches).

Dextrins like maltodextrin or yellow dextrin are examples of starch derivatives obtained by partial hydrolysis.

In a preferred process according to the present invention the starches or starch derivatives are chosen from the group consisting of amylopectin, OSA starches, maltodextrin and pregelatinised starches.

A preferred process according to the present invention is a powder catch process. Such a process is known from the prior art (for example from WO04062382). As a result of such a powder catch process the beadlets are covered by a layer of the powder.

Therefore, the beadlets produced according to this process are preferably covered by a layer of the powder catch medium. This layer (coating) is in the form of a powder coating. The powder catch medium is a compound (or a mixture of compounds), which is able to absorb moisture and to form a powder coating. Suitable powder catch media are i.e. starches, silicate or phosphate compounds. Preferred powder catch media are starches (such as i.e. corn starch), calcium silicate, calcium aluminum silicate and tri-calcium phosphate. Most preferred are starches, especially corn starch.

Beadlets are a well known form of formulation for fat-soluble substances. An important advantage of the generally spherical beadlets is that they are not dusty and that they posses excellent free flowing characteristics, which are very desirable for manufacturing and formulating operations.

Usually the size of a beadlet is from 50 μm to 1,000 μm (preferably from 250 μm to 850 μm). The sizes can be smaller or larger. The size of a beadlet can be determined according to well known methods, such as (scanning) electron microscopy.

A suitable method to produce beadlets as disclosed and described above is for example described in WO 2004/062382. This reference is hereby incorporated.

The process according to the present invention surprisingly allows producing beadlets with a high amount of water-dispersible probiotic bacteria with good overall properties.

The process as described in the present patent application can be used not only to produce beadlets with a high amount of probiotic compounds, but also with a low amount thereof. The amount can be as low as 1 wt-%, based on the total weight of the beadlets. Usually the content of the probiotic compounds in the beadlets is at least 5 wt-%, preferably at least 10 wt-%, based on the total weight of the beadlets.

A preferred embodiment of the present invention relates to a process as described above wherein the beadlets comprise at least 30 wt-%, based on the total weight of the beadlets, of at least one probiotic compound.

A further preferred embodiment of the present invention relates to a process as described above wherein the beadlets comprise up to 85 wt-%, based on the total weight of the beadlets, of at least one probiotic compound.

A preferred embodiment of the present invention relates to a process wherein the beadlets comprise up to 80 wt-%, based on the total weight of the beadlets, of at least one probiotic compound.

A preferred embodiment of the present invention relates to a process wherein the beadlets comprise at least 5 wt-%, based on the total weight of the beadlets, more preferred 20 wt-%, of at least one starch and/or at least one starch derivative (matrix material) and of the powder coating layer.

A preferred embodiment of the present invention relates to a process wherein the beadlets comprise

at least 5 wt-%, based on the total weight of the beadlets, of powder coating layer.

A more preferred embodiment of the present invention relates to a process wherein the beadlets comprise
(i) 5 wt-%-80 wt-%, preferably 30 wt-%-80 wt-%, more preferably 40 wt-%-70 wt-%, based on the total weight of the beadlets, of at least one probiotic compound, and
(ii) 5 wt-%-90 wt-%, preferably 25 wt-%-50 wt-%, based on the total weight of the beadlets, of at least one starch and/or at least one starch derivative, and
(iii) 5 wt-%-50 wt-%, preferably 5 wt-%-20 wt-%, based on the total weight of the beadlets, of powder coating.

An especially embodiment of the present invention relates to a process, wherein the starch component (ii), always comprise maltodextrin and at least one further starch and/or at least one further starch derivate.

The matrix of the beadlets of the present invention as described above can also comprise additional compounds. Such compounds can be any kind of auxiliaries used in the field of beadlet producing and/or food and feed technology. A preferred compound is sugar (sucrose).

A further embodiment of the present invention relates to a process for preparing beadlets, which comprise at least one probiotic compound, comprising:
(a) forming an aqueous solution of
   (i) at least one probiotic compound and
   (ii) at least one protein,
(b) converting the solution into a dry powder by spray drying into a collecting powder.

The probiotic compound is as defined above.

Proteins are large organic compounds made of amino acids arranged in a linear chain and joined together by peptide bonds between the carboxyl and amino groups of adjacent amino acid residues.

All living tissues (animals, human and plants) contain proteins.

Proteins are available as isolates, concentrates, meals and hydrolysates. Protein isolates contain more than 90% protein. Protein concentrates contain 60-90% protein. Protein meals contain less than 60% protein. In protein hydrolysates, a part of the peptide bonds is broken either chemically, e.g. by acid or enzymatically. Thereby the molecular weight and the degree of polymerisation of the protein is reduced. The amount of hydrolysis is described by the degree of hydrolysis (DH). The DH is the percentage of broken peptide bonds compared to all peptide bonds. Therefore a DH of 0% describes a native protein while a DH of 100% describes a completely hydrolysed protein.

The protein content in plants is very small. In contrast to plants, animal and human bodies are composed largely of proteins.

Sources of plant proteins are for example peas, beans (such as soya beans, castor beans, etc), lupins, cotton, potatoes, sweet potatoes, manioc, rapeseed, sunflowers, sesame, linseed, safflower, lentils, nuts, wheat, rice, maize, barley, rye, oats and sorghum.

Sources of animal proteins are for example blood, bones, skin, milk and eggs.

Sources of microbial proteins are for example bacteria or yeasts.

For the invention of the present patent application the following proteins are preferred: gelatines (especially fish and poultry), hydrolysed gelatines and milk proteins (casein, whey proteins).

Besides native protein isolates, concentrates or meals, proteins can be modified either mechanically, thermally, chemically or enzymatically.

Partial denaturation of the protein e.g. by heat treat may be used to improve the gel-forming properties of the protein. The protein may also be crosslinked e.g. enzymatically by transglutaminase.

To improve solubility or viscosity, the protein may be partially hydrolysed either chemically or enzymatically by proteases.

In the context, the term “proteins” includes native proteins as isolates, concentrates or meals as well as modified, denatured or crosslinked proteins or hydrolysates.

Therefore the present invention also relates to a process as described above, wherein the protein is a protein isolate, concentrate or meal.

The present invention also relates to a process as described above, wherein the protein is a protein hydrolysate.

The present invention also relates to a process as described above, wherein the protein is a heat-treated.

The present invention also relates to a process as described above, wherein the protein is crosslinked.

Therefore a further embodiment of the present invention relates to process for the production of beadlets, which comprise at least one probiotic compound and at least one protein extracted from a plant (such as peas, soya beans, castor beans, lupins, cotton, potatoes, sweet potatoes, manioc, rapeseed, sunflowers, sesame, linseed, safflower, lentils, nuts, wheat, rice, maize, barley, rye, oats and sorghum), an animal source (such as blood, bones, skin, milk and eggs) or a microbial source (such as bacteria or yeasts).

A preferred embodiment according to the present invention relates to the production of beadlets, which comprise at least one probiotic compound and at least one protein compounds chosen from the group consisting of gelatines, hydrolysed gelatines and milk protein.

The matrix of the beadlets of the present invention can also comprise additional compounds, such as sugar and/or maltodextrin.

Sugar refers to any monosaccharide or disaccharide (preferred is sucrose).

A preferred process according to present invention relates to a process wherein the beadlets comprise at least 5 wt-%, based on the total weight of the beadlets, more preferred 20 wt-%, of at least one protein (matrix material) and of the powder coating layer.

A preferred process according to present invention relates to a process wherein the beadlets comprise

at least 5 wt-%, based on the total weight of the beadlets, of powder coating layer.

A more preferred process according to present invention relates to a process wherein the beadlets comprise
(i) 5 wt-%-80 wt-%, preferably 30 wt-%-80 wt-%, more preferably 40 wt-%-70 wt-%, based on the total weight of the beadlets, of at least one probiotic compound, and
(ii) 5 wt-%-90 wt-%, preferably 25 wt-%-50 wt-%, based on the total weight of the beadlets, of at least one protein, and
(iii) 5 wt-%-50 wt-%, preferably 5 wt-%-20 wt-%, based on the total weight of the beadlets, of powder coating.

As mentioned above the matrix of the beadlets of the present invention can also comprise additional compounds. A preferred compound is sugar (sucrose).

A further embodiment of the present invention relates to a process for preparing beadlets, which comprise at least one probiotic compound, comprising:
(a) forming an aqueous solution of
   (i) at least one probiotic compound and
   (ii) at least one gum compound,
(b) converting the solution into a dry powder by spray drying into a collecting powder.

The probiotic compound is as defined above.

The term gum in the context of the present invention is defined as polysaccharides. Polysaccharides made up of only one type of neutral monosaccharide structural unit and with only one type of glycosidic linkage—as in cellulose or amylose—are denoted as perfectly linear polysaccharides. In branched polysaccharides the frequency of branching sites and the length of the side chains can vary greatly. Molecules with a long “backbone” chain and many short side chains are called linearly branched polysaccharides.

Polysaccharides are water soluble or swell in water, giving colloidal, highly viscous solutions or dispersions with plastic or pseudoplastic flow properties. Functional properties such as thickening, water holding and binding, stabilization of suspensions and emulsions, and gelling, are based on this behaviour. Therefore, polysaccharides are often referred to as gelling or thickening agents, stabilizers, water binders, or fillers.

Examples of gums are agar, alginate, arabinoxylan, carrageenan, chitosan, gellan, curdlan, β-glucan, guar gum, gum arabic (=gum acacia), locust bean gum, pectin, and xanthan gum.

The matrix of the beadlets of the present invention can also comprise additional compounds, such as maltodextrin and/or sugar (sucrose). A preferred combination of matrix materials is the combination of maltodextrin and gums.

Maltodextrin and gum arabic as matrix materials is an especially preferred embodiment of the present invention.

A preferred process according to present invention relates to a process, wherein the beadlets comprise at least 5 wt-%, based on the total weight of the beadlets, more preferred 20 wt-%, of at least one gum compound (matrix material) and of the powder coating layer.

A preferred process according to present invention relates to a process wherein the beadlets comprise

at least 5 wt-%, based on the total weight of the beadlets, of powder coating layer.

A more preferred process according to present invention relates to a process wherein the beadlets comprise
(i) 5 wt-%-80 wt-%, preferably 30 wt-%-80 wt-%, more preferably 40 wt-%-70 wt-%, based on the total weight of the beadlets, of at least one probiotic compound, and
(ii) 5 wt-%-90 wt-%, preferably 25 wt-%-50 wt-%, based on the total weight of the beadlets, of at least one gum compound, and
(iii) 5 wt-%-50 wt-%, preferably 5 wt-%-20 wt-%, based on the total weight of the beadlets, of powder coating.

As mentioned above the matrix of the beadlets of the present invention can also comprise additional compounds. A preferred combination of matrix materials is the combination of maltodextrin and gums. Maltodextrin and gum arabic is an especially preferred embodiment of the present invention. The ratio of maltodextrin:gum arabic can be 5:1 to 1:5.

A further embodiment of the present invention relates to a process for preparing beadlets, which comprise at least one probiotic compound, comprising:
(a) forming an aqueous solution of
   (i) at least one probiotic compound and
   (ii) at least one synthetic and/or semi-synthetic polymer,
(b) converting the solution into a dry powder by spray drying into a collecting powder.

The probiotic compound is as defined above

Synthetic polymers in the context of the present invention are defined as polymers chemically manufactured from separate materials. Polymers are macromolecules, composed of repeating structural units (monomers), which are connected by covalent chemical bonds. Synthetic polymers can be obtained from one or more monomers by polymerisation reactions like poly-addition, poly-condensation or anionic or cationic polymerisation. Most synthetic polymers are used for non-food applications like plastics or elastomers, but some are also approved for use in food, feed or pharmaceutical applications.

Preferred synthetic polymers are polyethylene glycols (PEG) and polyvinyl pyrrolidone.

Semi-synthetic polymers in the context of the present invention are obtained from polymers of natural origin by chemical modification. Preferred semi-synthetic polymers are based on cellulose or lignin as macromolecular backbone.

Semi-synthetic polymers comprise ethers and esters of cellulose or lignin, like carboxymethyl cellulose, cellulose acetate phthalate, hydroxypropyl-methyl cellulose, ethyl cellulose, methyl cellulose or lignosulfonates.

The matrix of the beadlets of the present invention can also comprise additional compounds, such as sugar and/or maltodextrin.

Sugar refers to any monosaccharide or disaccharide (preferred is sucrose).

A preferred process according to present invention relates to a process wherein the beadlets comprise at least 5 wt-%, based on the total weight of the beadlets, more preferred 20 wt-%, of at least one synthetic and/or semi-synthetic polymer (matrix material) and of the powder coating layer.

A preferred process according to present invention relates to a process wherein the beadlets comprise

at least 5 wt-%, based on the total weight of the beadlets, of powder coating layer.

A more preferred process according to present invention relates to a process wherein the beadlets comprise
(i) 5 wt-%-80 wt-%, preferably 30 wt-%-80 wt-%, more preferably 40 wt-%-70 wt-%, based on the total weight of the beadlets, of at least one probiotic compound, and
(ii) 5 wt-%-90 wt-%, preferably 25 wt-%-50 wt-%, based on the total weight of the beadlets, of at least one synthetic and/or semi-synthetic polymer, and
(iii) 5 wt-%-50 wt-%, preferably 5 wt-%-20 wt-%, based on the total weight of the beadlets, of powder coating.

As mentioned above the matrix of the beadlets of the present invention can also comprise additional compounds. A preferred compound is sugar (sucrose) and/or maltodextrin.

Beadlets comprising a high amount (at least 30 wt-%) of probiotic compounds in a matrix comprising starch(es) and/or at least one starch derivative(s) are not known from the prior art.

A further embodiment of the present invention relates to beadlets (B1) comprising
(i) at least 30 wt-%, based on the total weight of the beadlets, of at least one probiotic compound and
(ii) at least one starch and/or at least one starch derivative.

The invention also relates to beadlets (B2) comprising
(i) up to 85 wt-%, based on the total weight of the beadlets, of at least one probiotic compound, and
(ii) at least one starch and/or at least one starch derivative.

Preferred beadlets (B1′) according to present invention comprise
(i) at least 30 wt-%, based on the total weight of the beadlets, of at least one probiotic compound and
(ii) up to 70 wt-%, based on the total weight of the beadlets, of at least one starch and/or at least one starch derivative.

Further preferred beadlets (B2′) according to the present invention comprise
(i) up to 85 wt-%, based on the total weight of the beadlets, of at least one probiotic compound, and
(ii) at least 15 wt-%, based on the total weight of the beadlets, of at least one starch and/or at least one starch derivative.

Preferred starch covered beadlets according to present invention comprise at least 5 wt-%, based on the total weight of the beadlets, of the powder coating layer.

Therefore (B1), (B1′), (B2) and (B2′) preferably comprise 5 wt-%, based on the total weight of the beadlets, of powder coating layer.

More preferred beadlets according to the present invention (B3) comprise
(i) 30 wt-%-80 wt-%, preferably 40 wt-%-70 wt-%, based on the total weight of the beadlets, of at least one probiotic compound chosen from the group consisting of Bacillus, Lactobacillus, Pediococcus and Propionibacterium, and
(ii) 15 wt-%-65 wt-%, preferably 25 wt-%-50 wt-%, based on the total weight of the beadlets, of at least one starch and/or at least one starch derivative chosen from the groups consisting of corn starch, sorghum starch, wheat starch, rice starch, tapioca starch, arrowroot starch, sago starch, potato starch, quinoa starch and amaranth starch, pregelatinised starches, acidic modified starches, oxidized starches, cross-linked starches, starch esters, starch ethers and cationic starches (preferred are starches with a high amount of amylopectin, OSA starches, (malto)dextrins and pregelatinised starches), and
(iii) 5 wt-%-50 wt-%, preferably 5 wt-%-20 wt-%, based on the total weight of the beadlets, of powder coating chosen from the group consisting of starches (preferably corn starch), silicate and phosphate compounds.

In a more preferred embodiment of the present invention the starches or starch derivatives are chosen from the group consisting of amylopectin, OSA starches, maltodextrin and pregelatinised starches.

Especially preferred beadlets according to the present invention (B3′) comprise
(i) 5 wt-%-50 wt-%, based on the total weight of the beadlets, of at least one probiotic compound chosen from the group consisting of acidipropionici and P. jensenii (preferably P. acidipropionici and jensenii strains P169, P170, P179, P195, and P261, especially preferably strain P169), and
(ii) 15 wt-%-65 wt-%, based on the total weight of the beadlets, of at least one starch and/or at least one starch derivative chosen from the groups consisting of corn starch, sorghum starch, wheat starch, rice starch, tapioca starch, arrowroot starch, sago starch, potato starch, quinoa starch and amaranth starch, pregelatinised starches, acidic modified starches, oxidized starches, cross-linked starches, starch esters, starch ethers, dextrins and cationic starches (preferred are starches with a high amount of amylopectin, OSA starches, (malto)dextrins and pregelatinised starches), and
(iii) 5 wt-%-50 wt-%, based on the total weight of the beadlets, of powder coating chosen from the group consisting of starches (preferably corn starch), silicate and phosphate compounds.

Most preferred beadlets according to the present invention (B3″) comprise
(i) 40 wt-%-70 wt-%, based on the total weight of the beadlets, of at least one probiotic compound chosen from the group consisting of acidipropionici and P. jensenii (preferably P. Acidipropionici and jensenii strains P169, P170, P179, P195, and P261, especially preferably strain P169), and
(ii) 25 wt-%-50 wt-%, based on the total weight of the beadlets, of at least one starch and/or at least one starch derivative chosen from the groups consisting of corn starch, sorghum starch, wheat starch, rice starch, tapioca starch, arrowroot starch, sago starch, potato starch, quinoa starch and amaranth starch, pregelatinised starches, acidic modified starches, oxidized starches, cross-linked starches, starch esters, starch ethers, dextrins and cationic starches (preferred are starches with a high amount of amylopectin, OSA starches, (malto)dextrins and pregelatinised starches), and
(iii) 5 wt-%-20 wt-%, based on the total weight of the beadlets, of powder coating chosen from the group consisting of starches (preferably corn starch), silicate and phosphate compounds.

Beadlets comprising a high amount (at least 30 wt-%) of probiotic compounds in a matrix comprising protein(s) are not known from the prior art.

A further embodiment of the present invention relates to beadlets (B4) comprising
(i) at least 30 wt-%, based on the total weight of the beadlets, of at least one probiotic compound and
(ii) at least one protein.

The invention also relates to beadlets (B5) comprising
(i) up to 85 wt-%, based on the total weight of the beadlets, of at least one probiotic compound, and
(ii) at least one protein.

Preferred beadlets (B4′) according to present invention comprise
(i) at least 30 wt-%, based on the total weight of the beadlets, of at least one probiotic compound and
(ii) up to 70 wt-%, based on the total weight of the beadlets, of at least protein.

The beadlets (B5′) comprise
(i) up to 85 wt-%, based on the total weight of the beadlets, of at least one probiotic compound, and
(ii) at least 15 wt-%, based on the total weight of the beadlets, of at least one protein.

The beadlets (B5″) comprise
(i) up to 85 wt-%, based on the total weight of the beadlets, of at least one probiotic compound, and
(ii) at least 15 wt-%, based on the total weight of the beadlets, of gelatine and sucrose.

Preferred starch covered beadlets according to present invention comprise at least 5 wt-%, based on the total weight of the beadlets, of the power coating layer.

Therefore (B4), (B4′), (B5), (B5′) and (B5″) preferably comprise 5 wt-%, based on the total weight of the beadlets, of powder coating layer.

More preferred beadlets according to the present invention (B6) comprise
(i) 30 wt-%-80 wt-%, preferably 40 wt-%-70 wt-%, based on the total weight of the beadlets, of at least one probiotic compound chosen from the group consisting of Bacillus, Lactobacillus, Pediococcus and Propionibacterium, and
(ii) 15 wt-%-65 wt-%, preferably 25 wt-%-50 wt-%, based on the total weight of the beadlets, of at least one protein from a plant source (such as peas, soya, castor beans, lupins, cotton, potatoes, sweet potatoes, manioc, rapeseed, sunflowers, sesame, linseed, safflower, lentils, nuts, wheat, rice, maize, barley, rye, oats and sorghum) or an animal source (such as blood, bones, skin, milk and eggs) or a microbial source (such as bacteria and yeasts) and
(iii) 5 wt-%-50 wt-%, preferably 5 wt-%-20 wt-%, based on the total weight of the beadlet, of powder coating chosen from the group consisting of starches (preferably corn starch), silicate and phosphate compounds.

In more preferred process according to the present invention the proteins are chosen from the group consisting of gelatines (especially fish gelatines or poultry gelatines), hydrolysed gelatines and milk proteins (whey protein, casein).

Especially preferred beadlets according to the present invention (B6′) comprise
(i) 30 wt-%-80 wt-%, based on the total weight of the beadlets, of at least one probiotic compound chosen from the group consisting of acidipropionici and P. jensenii (preferably P. Acidipropionici and jensenii strains P169, P170, P179, P195, and P261, especially preferably strain P169), and
(ii) 15 wt-%-65 wt-%, based on the total weight of the beadlets, of at least one protein chosen from the group consisting of gelatines (especially fish gelatines), hydrolysed gelatines and milk proteins (whey protein, casein), and
(iii) 5 wt-%-50 wt-%, based on the total weight of the beadlets, of powder coating chosen from the group consisting of starches (preferably corn starch), silicate and phosphate compounds.

Most preferred beadlets according to the present invention (B6″) comprise
(i) 40 wt-%-70 wt-%, based on the total weight of the beadlets, of at least one probiotic compound chosen from the group consisting of acidipropionici and P. jensenii (preferably P. Acidipropionici and jensenii strains P169, P170, P179, P195, and P261, especially preferably strain P169), and
(ii) 25 wt-%-50 wt-%, based on the total weight of the beadlets, of at least one protein chosen from the group consisting of gelatines (especially fish gelatines), hydrolysed gelatines and milk proteins (whey protein, casein), and
(iii) 5 wt-%-20 wt-%, based on the total weight of the beadlets, of powder coating chosen from the group consisting of starches (preferably corn starch), silicate and phosphate compounds.

Beadlets comprising a high amount (at least 30 wt-%) of probiotic compounds in a matrix comprising gum compounds(s) are not known from the prior art.

Therefore, a further embodiment of the present invention relates to beadlets (B7) comprising
(i) at least 30 wt-%, based on the total weight of the beadlets, of at least one probiotic compound and
(ii) at least one gum compound.

The invention also relates to beadlets (B8) comprising
(iii) up to 85 wt-%, based on the total weight of the beadlets, of at least one probiotic compound, and
(iv) at least one gum compound.

Preferred beadlets (B7′) according to present invention comprise
(i) at least 30 wt-%, based on the total weight of the beadlets, of at least one probiotic compound and
(ii) up to 70 wt-%, based on the total weight of the beadlets, of at least one gum compound.

The beadlets (B8′) comprise
(iii) up to 85 wt-%, based on the total weight of the beadlets, of at least one probiotic compound, and
(iv) at least 15 wt-%, based on the total weight of the beadlets, of at least one gum compound.

The beadlets (B8″) comprise
(i) up to 85 wt-%, based on the total weight of the beadlets, of at least one probiotic compound, and
(ii) at least 15 wt-%, based on the total weight of the beadlets, of gum arabic and maltodextrin.

Preferred starch covered beadlets according to present invention comprise at least 5 wt-%, based on the total weight of the beadlets, of the power coating layer.

Therefore (B7), (B7′), (B8), (B8′) and (B8″) preferably comprise 5 wt-%, based on the total weight of the beadlets, of powder coating layer.

More preferred beadlets according to the present invention (B9) comprise
(i) 30 wt-%-80 wt-%, preferably 40 wt-%-70 wt-%, based on the total weight of the beadlets, of at least one probiotic compound chosen from the group consisting of Bacillus, Lactobacillus, Pediococcus and Propionibacterium, and
(ii) 15 wt-%-65 wt-%, preferably 25 wt-%-50 wt-%, based on the total weight of the beadlets, of at least one gum compound chosen from the groups consisting of agar, alginate, arabinoxylan, carrageenan, chitosan, gellan, curdlan, β-glucan, guar gum, gum arabic, locust bean gum, pectin, xanthan gum, and a combination of maltodextrin and gum arabic, and
(iii) 5 wt-%-50 wt-%, preferably 5 wt-%-20 wt-%, based on the total weight of the beadlets, of powder coating chosen from the group consisting of starches (preferably corn starch), silicate and phosphate compounds.

Most preferred beadlets according to the present invention (B9′) comprise
(i) 30 wt-%-80 wt-%, preferably 40 wt-%-70 wt-%, based on the total weight of the beadlets, of at least one probiotic compound chosen from the group consisting of acidipropionici and P. jensenii (preferably P. Acidipropionici and jensenii strains P169, P170, P179, P195, and P261, especially preferably strain P169), and
(ii) 15 wt-%-65 wt-%, preferably 25 wt-%-50 wt-%, based on the total weight of the beadlets, of at least one gum compound chosen from the groups consisting of agar, alginate, arabinoxylan, carrageenan, chitosan, gellan, curdlan, β-glucan, guar gum, gum arabic, locust bean gum, pectin, xanthan gum, and a combination of maltodextrin and gum arabic, and
(iii) 5 wt-%-50 wt-%, preferably 5 wt-%-20 wt-%, based on the total weight of the beadlets, of powder coating chosen from the group consisting of starches (preferably corn starch), silicate and phosphate compounds.

Further most preferred beadlets according to the present invention (B9″) comprise
(i) 30 wt-%-80 wt-%, preferably 40 wt-%-70 wt-%, based on the total weight of the beadlets, of at least one probiotic compound chosen from the group consisting of acidipropionici and P. jensenii (preferably P. Acidipropionici and jensenii strains P169, P170, P179, P195, and P261, especially preferably strain P 169), and
(ii) 15 wt-%-65 wt-%, preferably 25 wt-%-50 wt-%, based on the total weight of the beadlets, of gum arabic and maltodextrin, and
(iii) 5 wt-%-50 wt-%, preferably 5 wt-%-20 wt-%, based on the total weight of the beadlets, of powder coating chosen from the group consisting of starches (preferably corn starch), silicate and phosphate compounds.

Beadlets comprising a high amount (at least 30 wt-%) of probiotic compounds in a matrix comprising synthetic and/or semi-synthetic polymer(s) are not known from the prior art.

A further embodiment of the present invention relates to beadlets (B10) comprising
(i) at least 30 wt-%, based on the total weight of the beadlets, of at least one probiotic compound and
(ii) at least one synthetic and/or semi-synthetic polymer.

The invention also relates to beadlets (B11) comprising
(v) up to 85 wt-%, based on the total weight of the beadlets, of at least one probiotic compound, and
(vi) at least one synthetic and/or semi-synthetic polymer.

Preferred beadlets (B10′) according to present invention comprise
(i) at least 30 wt-%, based on the total weight of the beadlets, of at least one probiotic compound and
(ii) up to 70 wt-%, based on the total weight of the beadlets, of at least one synthetic and/or semi-synthetic polymer.

The beadlets (B11′) comprise
(v) up to 85 wt-%, based on the total weight of the beadlets, of at least one probiotic compound, and
(vi) at least 15 wt-%, based on the total weight of the beadlets, of at least one synthetic and/or semi-synthetic polymer.

The beadlets (B11″) comprise
(iii) up to 85 wt-%, based on the total weight of the beadlets, of at least one probiotic compound, and
(iv) at least 15 wt-%, based on the total weight of the beadlets, of at least one synthetic and/or semi-synthetic polymer.

Preferred starch covered beadlets according to present invention comprise at least 5 wt-%, based on the total weight of the beadlets, of the power coating layer.

Therefore (B10), (B10′), (B11), (B11′) and (B11″) preferably comprise 5 wt-%, based on the total weight of the beadlets, of powder coating layer.

More preferred beadlets according to the present invention (B12) comprise
(i) 30 wt-%-80 wt-%, preferably 40-70 wt-%, based on the total weight of the beadlets, of at least one probiotic compound chosen from the group consisting of Bacillus, Lactobacillus, Pediococcus and Propionibacterium, and
(ii) 15 wt-%-65 wt-%, preferably 25-50 wt-%, based on the total weight of the beadlets, of at least one synthetic and/or semi-synthetic polymer chosen from the group consisting of polyethylene glycol, carboxymethyl cellulose (CMC), carboxypropyl-methyl cellulose (HPMC), polyvinyl pyrrolidone and lignosulfonates,
(iii) 5 wt-%-50 wt-%, preferably 5-20 wt-%, based on the total weight of the beadlets, of powder coating chosen from the group consisting of starches (preferably corn starch), silicate and phosphate compounds.

Especially preferred beadlets according to the present invention (B12′) comprise
(i) 30 wt-%-80 wt-%, based on the total weight of the beadlets, of at least one probiotic compound chosen from the group consisting of acidipropionici and P. jensenii (preferably P. Acidipropionici and jensenii strains P169, P170, P179, P195, and P261, especially preferably strain P169), and
(ii) 15 wt-%-65 wt-%, based on the total weight of the beadlets, of at least one synthetic and/or semi-synthetic polymer chosen from the group consisting of polyethylene glycol, carboxymethyl cellulose (CMC), carboxypropyl-methyl cellulose (HPMC), polyvinyl pyrrolidone and lignosulfonates.
(iii) 5 wt-%-50 wt-%, based on the total weight of the beadlets, of powder coating chosen from the group consisting of starches (preferably corn starch), silicate and phosphate compounds.

Most preferred beadlets according to the present invention (B12″) comprise
(i) 40 wt-%-70 wt-%, based on the total weight of the beadlets, of at least one probiotic compound chosen from the group consisting of acidipropionici and P. jensenii (preferably P. Acidipropionici and jensenii strains P169, P170, P179, P195, and P261, especially preferably strain P169), and
(ii) 25 wt-%-50 wt-%, based on the total weight of the beadlets, of at least one synthetic and/or semi-synthetic polymer chosen from the group consisting of polyethylene glycol, carboxymethyl cellulose (CMC), carboxypropyl-methyl cellulose (HPMC), polyvinyl pyrrolidone and lignosulfonates
(iii) 5 wt-%-20 wt-%, based on the total weight of the beadlets, of powder coating chosen from the group consisting of starches (preferably corn starch), silicate and phosphate compounds.

Further more preferred beadlets are beadlets (B1), (B1′), (B2), (B2′), (B2″), (B3), (B3′), (B3″), (B4), (B4′), (B5), (B5′), (B5″), (B6), (B6′), (B6″), (B7), (B7′), (B8), (B8′), (B8″), (B9), (B9′), (B9″), (B10), (B10′), (B11), (B11′), (B11″), (B12), (B12′) and (B12″) additionally comprising sugar (sucrose). These beadlets (B13) comprise 5-25 wt-%, preferably 10-20 wt-%, based on the total weight of the beadlets, of sucrose.

An especially preferred embodiment of the present invention are beadlets which comprise a mixture of maltodextrin and at least one further starch and/or starch derivative in the matrix. Therefore a further embodiment (B14) of the present invention relates to beadlets (B1), (B1′), (B2), (B2′), (B2″), (B3), (B3′) and (B3″), in which the starch component (ii) comprises maltodextrin and at least one further starch and/or starch derivative.

A further embodiment according to the present invention relates to the use of the beadlets (B1), (B1′), (B2), (B2′), (B2″), (B3), (B3′), (B3″), (B4), (B4′), (B5), (B5′), (B5″), (B6), (B6′), (B6″), (B7), (B7′), (B8), (B8′), (B8″), (B9), (B9′), (B9″), (B10), (B10′), (B11), (B11′), (B11″), (B12), (B12′), (B12″), (B13) and (B14) in food products (for humans and/or animals), dietary supplements as well in the production of food products and dietary supplements.

Depending what kind of probiotic compounds and/or matrix material is used the food product is suitable for humans or animals. In some cases a food product could be consumed by humans and animals.

A preferred embodiment according to the present invention relates to the use of the beadlets (B1), (B1′), (B2), (B2′), (B2″), (B3), (B3′), (B3″), (B4), (B4′), (B5), (B5′), (B5″), (B6), (B6′), (B6″), (B7), (B7′), (B8), (B8′), (B8″), (B9), (B9′), (B9″), (B10), (B10′), (B11), (B11′), (B11″), (B12), (B12′), (B12″), (B13) and (B14) in food for ruminants, especially for cows.

Food products (for humans and/or animals) in the context of the present comprise liquid and solid food products as well as paste-like and or gel like. The food products comprise food for humans as well as for animals (especially ruminants, poultry and swine).

A dietary supplement, also known as food supplement or nutritional supplement, is a preparation intended to supply nutrients, such as vitamins, minerals, fatty acids or amino acids that are missing or are not consumed in sufficient quantity in a person's diet.

The food product can be in a ready-to-consume form that means a form, which is suitable to eat without further proceedings. But it is also possible that food product is a form, which needs further proceedings, like heating, dissolving, diluting, etc.

Suitable human food products can be drinks, soups, bars (cereal, chocolate), dairy products, etc.

Suitable animal food products (feed products) can be in any commonly used form.

Therefore a further embodiment of the present invention relates to human and animal food products and to human and animal dietary supplements comprising beadlets as described above.

A preferred embodiment of the present invention relates to food products for ruminants (especially cows) comprising beadlets as described above.

The beadlets according to the present invention can also be used in premixes for food products (for humans and/or animals) and for dietary supplements (for humans and/or animals).

Premixes are a convenient usage form for the food producers but are a critical medium for probiotics due to pH, ionic strength and water activity values, which can negatively affect viability of probiotic bacteria. But the beadlets according to the present invention eliminate (or at least strongly minimize) such problems.

A preferred embodiment of the present invention relates to the use of the beadlets according to the present invention for the use in premixes for food for ruminants (especially cows).

A further embodiment of the present invention is a premix for food products (for humans and/or animals) and for dietary supplements (for humans and/or animals) comprising beadlets according to the present invention. A preferred embodiment of the present invention relates to a premix for food products for ruminants (especially cows).

Functional ingredients like vitamins and trace elements are often added to food or feed products as well as to premixes.

The following examples serve to illustrate the invention. The percentages are expressed in weight percentages and the temperatures are degrees Celsius, if not otherwise defined.

### EXAMPLE 1: FORMULATION OF PROPIONIBACTERIA (P169) IN A MATRIX COMPRISING AMYLOPECTIN

1.5 g amylopectin were added to 25 ml of water. The mixture was heated and stirred until dissolution occurred. The pH of the solution was adjusted to 7.0. To this solution, 50 g of concentrated biomass of propionibacteria (20% dry matter) were added under stirring. About 75 g of the suspension was sprayed in a spraying pan in a bed of fluidized starch at about 5° C. by means of a rotating spraying nozzle. The so-obtained beadlets were separated from excess starch by sieving and dried. There were obtained ca. 30 g of dry powder having an activity of 4.6 E11 cfu/g.

### EXAMPLE 2: PROBIOTIC FORMULATION WITH MALTODEXTRIN AND OSA-STARCH AS MATRIX

4.66 kg maltodextrin and 0.53 kg OSA-starch were dissolved in 35 kg concentrated biomass of propionibacteria (15% dry matter). The suspension was sprayed together with fluidized starch using a pilot-scale beadlet tower. The so-obtained beadlets were separated from excess starch and dried in an external fluid-bed. There were obtained ca. 13 kg of dry powder having an activity of 1.5 E11 cfu/g.

### EXAMPLE 3: PROBIOTIC FORMULATION WITH MALTODEXTRIN AS MATRIX

5.17 kg maltodextrin were dissolved in 35 kg concentrated biomass of propionibacteria (15% dry matter). The suspension was sprayed together with fluidized starch using a pilot-scale beadlet tower. The so-obtained beadlets were separated from excess starch and dried in an external fluid-bed. There were obtained ca. 13 kg of dry powder having an activity of 9.6 E10 cfu/g.

### EXAMPLE 4: PROPIONIBACTERIA (P169) IN A MATRIX COMPRISING FISH GELATINE AND SUGAR

40 g of fish gelatine and 20 g of sucrose were added to 80 ml of water. The mixture was stirred until dissolution occurred. The pH of the solution was adjusted to 7.0. To this solution, 60 g of concentrated biomass of propionibacteria (20% dry matter) were added under stirring. About 180 g of the suspension was sprayed in a spraying pan in a bed of fluidized starch at about 5° C. by means of a rotating spraying nozzle. The so-obtained beadlets were separated from excess starch by sieving and dried. There were obtained ca. 120 g of dry powder having an activity of 8.2 E10 cfu/g.

### EXAMPLE 5: PROPIONIBACTERIA (P169) IN A MATRIX COMPRISING GUM ARABIC AND MALTODEXTRIN

150 g concentrated biomass of propionibacteria (20% dry matter) were diluted with 50 g water. 52 g maltodextrin and 10 g gum arabic were added. The mixture was stirred until dissolution occurred. About 240 g of the suspension was sprayed in a spraying pan in a bed of fluidized starch at about 5° C. by means of a rotating spraying nozzle. The so-obtained beadlets were separated from excess starch by sieving and dried. There were obtained ca. 160 g of dry powder having a activity of 1.5 E11 cfu/g.

### EXAMPLE 6: PROPIONIBACTERIA (P169) IN A MATRIX COMPRISING POLYETHYLENE GLYCOL

5 g of polyethylene glycol PEG1000 were molten at 45° C. and added to 50 g concentrated biomass of propionibacteria (20% dry matter) with stirring. 5 g water was added. The suspension was sprayed in a spraying pan in a bed of fluidized starch at about 5° C. by means of a rotating spraying nozzle. The so-obtained beadlets were separated from excess starch by sieving and dried. There were obtained ca. 24 g of dry powder having an activity of 4.1 E11 cfu/g.

1. A process for preparing beadlets containing a high content of at least one probiotic, wherein the process comprises the steps of:
(a) forming an aqueous probiotic-containing solution consisting of:
(i) at least one probiotic in an amount of 40 wt. %-85 wt. %, based on the total weight of the beadlets, and
(ii) at least one starch and/or starch derivative, and thereafter
(b) converting the aqueous probiotic-containing solution into a dry powder by spray drying the solution into a fluidized bed of a collecting powder to form beadlets comprising the at least one probiotic, wherein
step (b) is practiced by the steps of:
(b1) dehumidifying hot atomizing air to a water content of less than about 3 g/kg to obtain hot dehumidified atomizing air;
(b2) atomizing the aqueous probiotic-containing solution into a spray zone of a spraying tower at a solution temperature of from about 15° C. to about 80° C. with the hot dehumidified atomizing air at a temperature sufficient to provide a spray zone temperature of about 60° C. to about 120° C.; while
(b3) fluidizing the fluidized bed of powder within a bottom of the spraying tower with cold fluidizing air at a temperature of between 5° C. to 20° C. at a ratio of hot atomizing air flow to cold fluidizing air flow in a range of 1:8 to 1:4 to achieve a temperature of the fluidized bed of powder of between about 5° C. to 20° C.,
(b4) controlling the temperature of the fluidized bed by controlling the supply and temperature of the cold fluidizing air, and
(b5) covering the beadlets by a powder coating layer comprised of at least one coating material selected from the group consisting of starches, calcium silicate, calcium aluminum silicate and tri-calcium phosphate so that the beadlets comprise at least 5 wt. %, based on the total weight of the beadlets, of the powder coating layer.

2. The process according to claim 1, wherein the probiotic is selected from the group consisting of Bacillus, Lactobacillus, Pediococcus and Propionibacterium.

3. The process according to claim 1, wherein the probiotic is selected from the group consisting of Propionibacterium Acidipropionici and jensenii strains P169, P170, P179, P195, and P261.

4. The process according to claim 1, wherein the starch and/or starch derivative is selected from the group consisting of corn starch, sorghum starch, wheat starch, rice starch, tapioca starch, arrowroot starch, sago starch, potato starch, quinoa starch and amaranth starch, pregelatinised starches, acidic modified starches, oxidized starches, cross-linked starches, starch esters, starch ethers, dextrins and cationic starches.

5. The process according to claim 1, wherein the starch and/or starch derivative is selected from the group consisting of amylopectin, OSA starches, maltodextrin and pregelatinised starches.

6. The process according to claim 1, wherein the starch and/or starch derivatives comprise a maltodextrin and at least one further starch and/or starch derivative.

7. (canceled)

8. The process according to claim 1, wherein the beadlets comprise up to 70 wt. %, based on the total weight of the beadlets, of the at least one probiotic.

9. The process according to claim 1, wherein the beadlets comprise up to 80 wt. %, based on the total weight of the beadlets, of the at least one probiotic.

10. The process according to claim 1, wherein the beadlets comprise at least 5 wt-%, based on the total weight of the beadlets, of the at least one starch and/or at least one starch derivative.

11. (canceled)
